(19) United States
(12) Patent Application Publication
(10) Pub. No.: US 2022/0317116 A1
(43) Pub. Date: Oct. 6, 2022

(54) A QUALITY DETECTION METHOD OF AN IMMUNOCHIP

(71) Applicant: BOE TECHNOLOGY GROUP CO., LTD., Beijing (CN)

(72) Inventors: Haonan LIU, Beijing (CN); Yudan YIN, Beijing (CN)

(21) Appl. No.: 17/600,301

(22) PCT Filed: Feb. 23, 2021
(86) PCT No.: PCT/CN2021/077447

§ 371 Date: Sep. 30, 2021

(30) Foreign Application Priority Data
Mar. 31, 2020 (CN) 202010245737.5

(51) Int. Cl. G01N 33/533

(52) U.S. Cl. CPC G01N 33/533

(57) ABSTRACT

A quality detection method of an immunochip is disclosed. At least part of a surface of the immunochip is a modified surface with modifying groups. The method comprises: applying a detection solution containing a labeled antibody on the modified surface of the immunochip, the labeled antibody is an antibody connected with a labeling substance, and the labeled antibody is capable of reacting with the modifying groups and bonding to the modifying groups; removing the labeled antibody that has not reacted with the modifying groups, and detecting the content of residual labeled antibody on the modified surface; and identifying the quality of the immunochip according to the content of residual labeled antibody.

### TECHNICAL FIELD

The present disclosure relates to the technical field of immunodiagnosis, in particular to a quality detection method of an immunochip.

### BACKGROUND

In recent years, immunochips (such as microfluidic chips) have gradually become a new type of immunodiagnostic consumables, which have the advantages of short reaction time, low reagent consumption, simple operation and high integration. The mainstream immunodiagnostic technologies using microfluidic chips are classified into immunofluorescence method, chemiluminescence method and electrochemiluminescence method. The immunofluorescence method is widely used because of its simple operation and economic advantages.

When immunofluorescence method is used for immunodiagnosis, it is necessary to carry out multi-layer biochemical modification on the immunochip. The effect of the modification will directly affect the quality and detection effect of the immunochip, so it is necessary to test the effect of the modification to ensure the quality and detection effect of the immunochip.

Using double-antibody sandwich method to test the modification effect leads to high labor costs and serious reagent waste; at the same time, the double-antibody sandwich method has many operating steps, and each additional operating step may cause errors in the test of the modification effect and affect the final detection effect.

### SUMMARY

A quality detection method of an immunochip is disclosed. At least part of a surface of the immunochip is a modified surface with modifying groups, and the method comprises:

applying a detection solution containing a labeled antibody on the modified surface of the immunochip, the labeled antibody is an antibody connected with a labeling substance, and the labeled antibody is capable of reacting with the modifying groups and bonding to the modifying groups;

removing the labeled antibody that has not reacted with the modifying groups, and detecting the content of residual labeled antibody on the modified surface; and

identifying the quality of the immunochip according to the content of residual labeled antibody.

Optionally, the modifying groups are carboxyl groups.

Further optionally, before applying the detection solution containing the labeled antibody on the modified surface of the immunochip, the method further comprises: performing an activation treatment on the immunochip for increasing the activity of the carboxyl groups modified on the modified surface of the immunochip.

Further optionally, the activity treatment to the immunochip comprises: placing the immunochip in an activation solution and keeping for a first predetermined time. The activation solution is a mixed solution of a 1-(3-dimethylaminopropyl)-3-ethylcarbodiimide hydrochloride solution and a N-hydroxysuccinimide solution.

Further optionally, the 1-(3-dimethylaminopropyl)-3-ethylcarbodiimide hydrochloride solution and the N-hydroxysuccinimide solution are mixed in a ratio of 1:0.8 to 1:1.2; wherein, the 1-(3-dimethylaminopropyl)-3-ethylcarbodiimide hydrochloride solution has a concentration of 6 wt % to 8 wt %; and the N-hydroxysuccinimide solution has a concentration of 1 wt % to 3 wt %.

Further optionally, the first predetermined time is 10 minutes to 20 minutes.

Further optionally, between applying the detection solution containing the labeled antibody on the modified surface of the immunochip and removing the labeled antibody that has not react with the modifying groups, the method further comprises: placing the immunochip in a predetermined environment and keeping for a second predetermined time.

Further optionally, the second predetermined time is greater than or equal to 8 hours.

Further optionally, the predetermined environment evades the light, and the predetermined environment has an environmental relative humidity of greater than 50%, and an environmental temperature of 2° C. to 6° C.

Optionally, the labeling substance is a fluorescent substance, and the labeled antibody is a fluorescent antibody; identifying the quality of the immunochip according to the content of residual labeling antibody comprises: identifying the quality of the immunochip according to the fluorescence intensity of the fluorescent substance of the residual fluorescent antibody.

Further optionally, identifying the quality of the immunochip according to the fluorescence intensity of the fluorescent substance of the residual fluorescent antibody comprises:

acquiring a fluorescence image of the immunochip;

randomly selecting a plurality of fluorescent regions of a predetermined area from the fluorescent image, and acquiring the fluorescence intensity value of each fluorescent region; and

acquiring an average value of the fluorescence intensity values of the plurality of fluorescent regions, and identifying the quality of the immunochip according to the average value.

Optionally, the modified surface includes a quality control area and an experimental area. Applying the detection solution containing the labeled antibody on the modified surface of the immunochip comprises applying the detection solution containing the labeled antibody on the quality control area.

### BRIEF DESCRIPTION OF THE FIGURES

The accompanying drawings are used to provide a further understanding of the embodiments of the present disclosure, and constitute a part of the specification, and are used to explain the present disclosure together with the embodiments of the present disclosure without a limitation to the present disclosure. The above and other features and advantages will become more apparent to those skilled in the art by describing detailed embodiments with reference to the accompanying drawings.

FIG. 1 is a flowchart of the quality detection method of an immunochip according to an embodiment of the present disclosure;

FIG. 2 is a schematic diagram of the dehydration condensation of amino group and carboxyl group;

FIG. 3 is a flowchart of the quality detection method of an immunochip according to another embodiment of the present disclosure;

FIG. 4 is a structural diagram of the immunochip according to an embodiment of the present disclosure;

FIG. 5 is an experimental diagram of randomly selecting a fluorescent region of a predetermined area from an image according to an embodiment of the present disclosure.

### DETAILED DESCRIPTION

In order to enable those skilled in the art to better understand the technical solutions of the present disclosure, the present disclosure will be further described in detail below with reference to the accompanying drawings and specific embodiments.

It is to be understood that the specific embodiments and drawings described herein are only used to explain the present disclosure, but not to limit the present disclosure.

It is to be understood that, in the case of no conflict, each embodiment in the present disclosure and each feature in the embodiment can be combined with each other.

It is to be understood that, for ease of description, only parts related to the present disclosure are shown in the drawings of the present disclosure, and parts not related to the present disclosure are not shown in the drawings.

It is to be understood that, in the case of no conflict, the functions and steps shown in the flowcharts and block diagrams of the present disclosure may occur in a different order from the order marked in the drawings.

An immunochip refers to a chip used for immunodiagnosis. In the preparation process of the immunochip, at least part of the surface of the immunochip needs to be biochemically modified, that is, the surface that needs to be modified is modified with modifying groups having certain properties. The surface of the immunochip upon biochemical modification has modifying groups, and this part of the surface is the modified surface of the immunochip.

The amount of modifying groups has a direct impact on the immunodetection effect of the immunochip, so it is necessary to detect the amount of modifying groups on the immunochip so as to ensure the immunodetection effect.

In some related technologies, the double-antibody sandwich method is used for detection. The double-antibody sandwich method specifically includes: connecting the antibody to the modifying group and incubating the same, and then reacting with the corresponding antigen of the antibody; after the reaction is completed, reacting the antigen with another labeled antibody; and indirectly detecting the modification effect of the modifying group by measuring the amount of the labeled antibody.

However, the above-mentioned double antibody sandwich method has many steps, and each additional operating step will cause errors in the test of the modification effect, and if the final modification effect is not good, a large amount of expensive reagents and labor costs will be wasted.

To this end, a method for detecting modifying groups on an immunochip is disclosed, which can detect the modifying groups on an immunochip quickly and effectively.

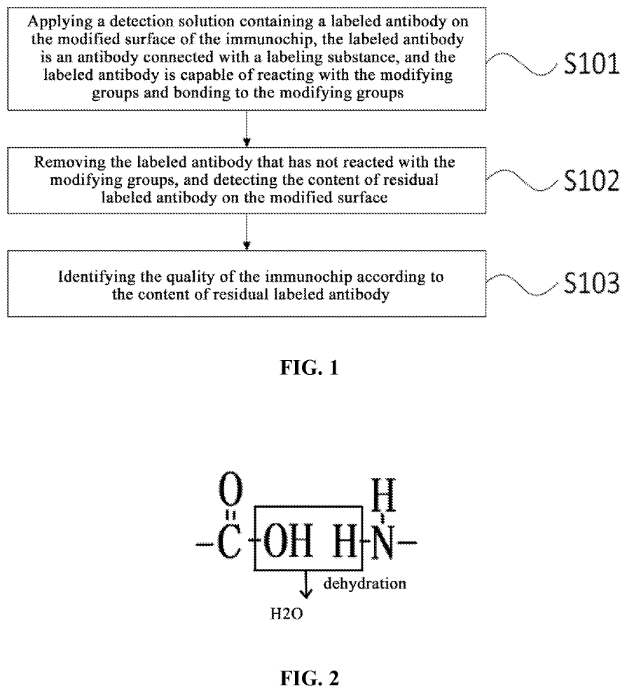

As shown in FIG. 1, an embodiment of the present disclosure provides a quality detection method of an immunochip. The method includes steps of S101, S102 and S103.

At S101, a detection solution containing a labeled antibody is applied on the modified surface of the immunochip. The labeled antibody is an antibody connected with a labeling substance, and the labeled antibody is capable of reacting with the modifying groups and bonding to the modifying groups.

The detection solution containing the labeled antibody is applied to the modified surface of the immunochip so as to allow the detection solution containing the labeled antibody to contact the modified surface.

The labeled antibody is an antibody to which a labeling substance is attached. The labeling substance refers to a substance that can be easily identified and qualitatively or quantitatively detected, and the labeled antibody can also react with the modifying groups on the modified surface and bond to the modifying groups.

After applying the detection solution containing the labeled antibody on the modified surface of the immunochip, the labeled antibody in the detection solution reacts with the modifying groups. The labeled antibody reacts with the modifying groups and bonds to the modifying groups, thereby staying on the modified surface.

At S102, the labeled antibody that has not reacted with the modifying groups is removed, and the content of residual labeled antibody on the modified surface is detected.

After removing the free labeled antibody that has not reacted with the modifying groups on the immunochip, the content of residual labeled antibody on the modified surface is detected (actually detecting the labeling substance).

At S103, the modification effect of the immunochip is identified according to the content of residual labeled antibody.

The modification effect of the immunochip is identified by the content of residual labeled antibody on the modified surface, that is, the amount of modifying groups on the surface of the immunochip.

The quality detection method of the immunochip according to the embodiment of the present disclosure utilizes a labeled antibody that can bind to the modifying groups, and indirectly detects the amount of the modifying groups by detecting the content of residual labeled antibody. The detection method of the present disclosure is simple to operate, reduces the detection steps of the modifying groups of the immunochip, reduces the influence of multiple steps on the detection results, increases the detection efficiency and improves the reliability of the detection results.

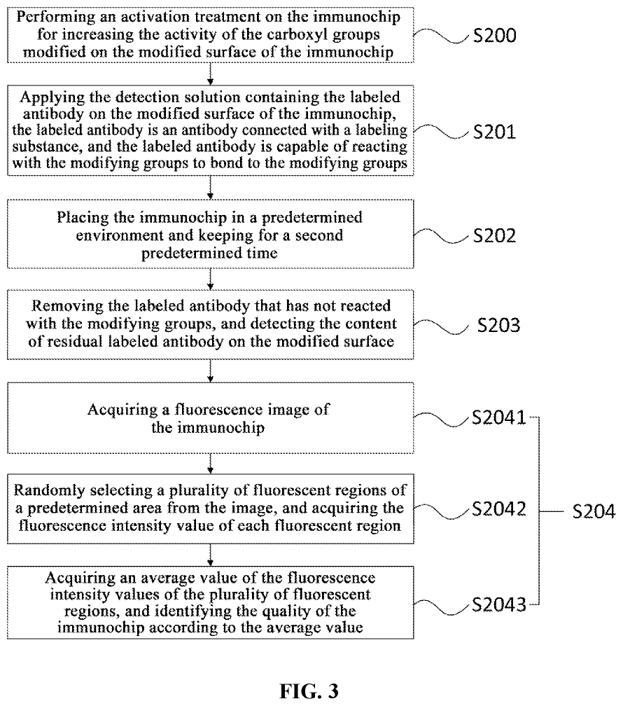

In one embodiment of the present disclosure, the modifying groups are carboxyl groups. As shown in FIG. 2, the carboxyl group may undergo dehydration condensation with the amino group, and the carboxyl group and the amino group are connected through an amide bond after the dehydration condensation. When the antibody (such as a labeled antibody) contains an amino group, the amino group may be dehydrated and condensed with the carboxyl group on the modified surface to connect the antibody (such as a labeled antibody) to the modified surface. Since the labeled antibody is also connected with a labeling substance, the labeling substance may be attached to the modified surface at the same time.

As shown in FIG. 3, another embodiment of the present disclosure provides a quality detection method of an immunochip. In this method, the modifying groups are carboxyl groups, and the labeled antibody contains an amino group. The method specifically includes steps of S200, S201, S202, S203 and S204.

At S200, an activation treatment is performed on the immunochip, and the activation treatment is used to increase the activity of the carboxyl groups modified on the modified surface of the immunochip.

The immunochip is activated before the detection of the immunochip. The purpose of the activation treatment is to increase the activity of the carboxyl groups on the modified surface of the immunochip.

After the immunochip is placed for a certain period of time, the activity of the carboxyl group may decrease, and the activity of the carboxyl group will directly affect the reaction between the carboxyl group and the amino group of the labeled antibody, thereby affecting the detection result. In practical applications, the detection is not always performed immediately after the surface modification of the immunochip, so the activation treatment of the immunochip can improve the accuracy of the detection.

Optionally, the activation treatment of the immunochip may specifically include placing the immunochip in an activation solution to react and keeping for a first predetermined time. The activation solution is a mixed solution of a 1-(3-dimethylaminopropyl)-3-ethylcarbodiimide hydrochloride (EDC) solution and a N-hydroxysuccinimide solution (NHS).

The immunochip is placed in a container (such as a petri dish), and the prepared EDC solution and NHS solution are poured into the petri dish at the same time to ensure that the mixed solution can submerge the immunochip, and the petri dish is placed on a horizontal shaker for activation.

After activation for a certain period of time (that is, the first predetermined time), the activated immunochip is taken out and rinsed with deionized water for 2 to 3 times, and then dried with nitrogen.

Further optionally, the EDC solution and the NHS solution are mixed in a ratio of 1:0.8 to 1:1.2; the EDC solution has a concentration of 6 wt % to 8 wt %; and the NHS solution has a concentration of 1 wt % to 3 wt %.

Further optionally, the first predetermined time is 10 minutes to 20 minutes.

When the activation solution is a mixed solution of EDC solution and NHS solution, the optimal activation time of the immunochip in the activation solution is 10 minutes to 20 minutes.

At S201, the detection solution containing the labeled antibody is applied on the modified surface of the immunochip. The labeled antibody is an antibody connected with a labeling substance, and the labeled antibody is capable of reacting with the modifying groups to bond to the modifying groups.

The detection solution containing the labeled antibody is applied on the modified surface of the immunochip that is modified with carboxyl groups, that is, the detection solution containing the labeled antibody can contact the modified surface.

Wherein, the labeled antibody is an antibody connected with a labeling substance and an amino group. The labeling substance refers to a substance that can be easily identified and qualitatively or quantitatively detected. The amino group of the labeled antibody can be dehydrated and condensed with the carboxyl group on the modified surface to make the labeled antibody connected to the carboxyl group. Since the carboxyl group is modified on the modified surface, the labeled antibody that reacts with the carboxyl group and is connected thereto is finally connected to the modified surface, and the labeling substance stays on the modified surface.

There are many ways to apply the detection solution containing the labeled antibody on the modified surface of the immunochip, such as placing the immunochip on the spotting instrument, keeping the environmental moist of the spotting instrument, and spotting the labeled antibodies on the immunochip according to the coordinates of the spotting platform array.

At S202, the immunochip is placed in a predetermined environment and keeping for a second predetermined time.

After applying the detection solution containing the labeled antibody, the immunochip is placed in a predetermined environment and keeping for a second predetermined time, so that the labeled antibody reacts with the carboxyl groups on the modified surface of the immunochip.

Further optionally, the second predetermined time is greater than or equal to 8 hours.

Further optionally, the predetermined environment evades the light, and the predetermined environment has an environmental relative humidity of greater than 50%, and an environmental temperature of 2° C. to 6° C.

Under the conditions of light-proof, environment relative humidity of greater than 50%, and temperature of 2° C. to 6° C., the carboxyl groups on the modified surface of the immunochip can react better with the labeled antibody in the detection solution, and the labeled antibody can be better connected to the modified surface.

At S203, the labeled antibody that has not reacted with the modifying groups is removed, and the content of residual labeled antibody on the modified surface is detected.

After removing the labeled antibody that has not reacted with the carboxyl group on the immunochip, the content of residual labeled antibody on the modified surface is detected (actually detecting the labeling substance).

The detection methods for the content of residual labeled antibody include, but are not limited to, fluorescence microscopy, laser confocal microscopy, ultraviolet-visible spectroscopy, gas chromatography, and liquid chromatography.

At S204, the quality of the immunochip is identified according to the content of residual labeled antibody.

The inspector can judge the quality of the immunochip by the content of residual labeled antibody on the modified surface.

Optionally, the labeling substance is a fluorescent substance, and the labeled antibody is a fluorescent antibody.

That is, the labeled antibody contains a fluorescent substance. Since the fluorescent substance is fluorescent under the light corresponding to the fluorescein, the labeled antibody can also be fluorescent under the light corresponding to the fluorescein of the fluorescent substance, thereby facilitating the detection. The quality of the immunochip can be identified based on the fluorescence intensity of the fluorescent substance of the residual fluorescent antibody.

For example, the rinsed and dried immunochip is placed under a fluorescence microscope, and the excitation light of the corresponding wavelength is selected according to the fluorescein used in the labeled antibody. The labeled antibody on the spotting position of the immunochip is placed under the excitation light. If the position is modified with carboxyl groups, the modified carboxyl groups will be connected to the labeled antibody due to the reaction, so that the corresponding fluorescence can be observed under a fluorescence microscope. The amount of carboxyl groups modified on the immunochip can be identified according to the fluorescence intensity, thereby identifying the quality of the immunochip.

The fluorescent substance is easy to label and easy to observe, so the fluorescent substance as a labeling substance can improve the efficiency of detection.

As shown in FIG. 3, in an embodiment of the present disclosure, identifying the quality of the immunochip based on the fluorescence intensity of the fluorescent substance of the residual fluorescent antibody includes steps of S2041, S2042 and S2043.

At S2041, a fluorescence image of the immunochip is acquired.

The fluorescence image of the immunochip is acquired under the fluorescence microscope. Since only the labeled antibody will emit light under the fluorescence microscope, the fluorescent position in the fluorescence image corresponds to the position where the fluorescent antibody is connected to the immunochip.

At S2042, a plurality of fluorescent regions of a predetermined area are randomly selected from the image, and the fluorescent intensity value of each fluorescent region is acquired.

At S2043, an average value of the fluorescence intensity values of the plurality of fluorescence regions is acquired, and the quality of the immunochip is identified according to the average value.

A plurality of fluorescent regions of a predetermined area are selected from the fluorescent image, and fluorescence intensity values of these fluorescent regions are obtained. According to the average value of the fluorescent intensity values of the plurality of fluorescent regions, the efficiency of the labeled antibody connecting to the carboxyl group can be identified, which indirectly reflects the modification effect of the carboxyl modifying group. The larger the average value of the fluorescence intensity, the better the carboxyl modification effect, that is, the higher the quality of the immunochip.

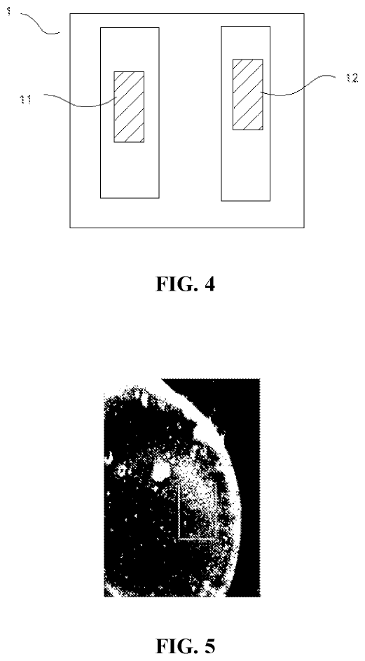

As shown in FIG. 4, in another embodiment of the present disclosure, the modified surface of the immunochip 1 includes a quality control area 11 and an experimental area 12, and the quality control method of the immunochip provided in the embodiment of the present disclosure is implemented in the quality control area 11.

The modified surface of the immunochip includes two different areas, namely the quality control area 11 and the experimental area 12. These two areas are modified with the same content of modifying groups. The quality detection method of the immunochip of the present disclosure can be implemented in the quality control area 11 firstly to detect the modification effect of the modifying groups of the immunochip quickly and simply. When the test result of the quality control area 11 meets certain requirements, it indicates that the quality of the modification effect reaches the standard and the immunochip is qualified, and then a normal immunological diagnosis can be performed in the experimental area 12. Each immunochip may be put into practical application after being tested. This method not only ensures the quality of the immunochip put into practical application, but also reduces the consumption of reagents and improves the efficiency of immunodiagnosis.

### EXAMPLES

### Example 1: Activation of Immunochip with Modifying Group of Carboxyl Group

First, 1.53 g of EDC was dissolved in 20 mL of deionized water, and was shaken and mixed for later use. 0.23 g of NHS was dissolved in 20 mL of deionized water, and was shaken and mixed for later use. Then, the as-prepared immunochip with modifying group of carboxyl group was placed in a petri dish, and the prepared EDC solution and NHS solution were poured into the petri dish at the same time to ensure that the immunochip was completely immersed in the solution. Then, the petri dish was placed on a horizontal shaker to react and activate for 15 minutes. Then, the activated immunochip was taken out, rinsed with deionized water for 3 times, and dried with nitrogen.

### Example 2: Spotting and Incubation of Immunochip

The activated immunochip in Example 1 was placed on the spotting instrument, which was kept at the ambient humidity of 60%. The detection solution containing the labeled antibody was spotted on the immunochip according to the coordinates of the spotting platform array.

A dust-free paper (or a dust-free cloth) was laid on the bottom of the petri dish and wetted with deionized water (to keep the immunochip in a humid environment during the incubation process). Then, the spotted immunochip was placed on the moist dust-free paper (or dust-free cloth). The petri dish was sealed with parafilm, and the petri dish was wraped with aluminum foil to evade the light. The processed petri dish was kept at 4° C. overnight.

### Example 3: Fluorescence Detection and Data Processing of Immunochip

The immunochip processed in Example 2 was rinsed 3 times with phosphate buffer solution, then rinsed 3 times with deionized water, and then dried with nitrogen. The purpose of rinse is to remove non-specifically adsorbed labeled antibodies, that is, labeled antibodies that have not reacted with carboxyl groups.

The immunochip was placed under a fluorescence microscope, and the fluorescence images were took under excitation light conditions.

As shown in FIG. 5, the image was process with software. 20 fluorescent regions with an area of 48,567 pixels were selected from the fluorescent image, and the fluorescent intensity value of each fluorescent region was acquired, so as to calculate the average value of the fluorescent intensity value. The specific data are shown in Table 1 below. Wherein, the number of times indicates which number of times the data has been acquired, the area is the area pixel value of the selected area at this time, and the fluorescence intensity is the fluorescence intensity value of the selected area at this time.

TABLE 1
Calculation of the average fluorescence
intensity value from the fluorescence image

| | | fluorescence |
|---|---|---|
| Times | Area | intensity |
| 1 | 48567 | 25.774 |
| 2 | 48567 | 25.741 |
| 3 | 48567 | 25.586 |
| 4 | 48567 | 25.632 |
| 5 | 48567 | 25.571 |
| 6 | 48567 | 25.47 |
| 7 | 48567 | 25.948 |
| 8 | 48567 | 25.006 |
| 9 | 48567 | 25.969 |
| 10 | 48567 | 25.917 |
| 11 | 48567 | 25.575 |
| 12 | 48567 | 25.869 |
| 13 | 48567 | 25.927 |
| 14 | 48567 | 25.988 |
| 15 | 48567 | 25.896 |
| 16 | 48567 | 25.855 |
| 17 | 48567 | 25.958 |
| 18 | 48567 | 25.203 |
| 19 | 48567 | 25.475 |
| 20 | 48567 | 25.539 |
| Average | 48567 | 25.745 |
| value | | |

The data in Table 1 show that the method of the present disclosure can detect the modification effect of the modifying groups of the immunochip, thereby identifying the quality of the immunochip.

The quality detection method of the immunochip of the embodiment of the present disclosure utilizes the labeled antibody that can bind to the modifying groups to indirectly detect the modification effect of the modifying groups by detecting the content of the labeled antibody introduced by the reaction. The method of the present disclosure is simple to operate, reduces the detection steps of the immunochip, increases the detection efficiency and reduces the impact of multiple steps on the detection results, thereby improving the reliability of the detection results.

It should be understood that the above embodiments are merely exemplary implementations adopted to illustrate the principle of the present application, and the present application is not limited thereto. For those of ordinary skill in the art, various modifications and improvements can be made without departing from the spirit and essence of the present application, and these modifications and improvements are also deemed to fall within the protection scope of the present application.

1. A quality detection method of an immunochip, at least part of a surface of the immunochip being a modified surface with modifying groups, wherein the method comprises the following steps:
applying a detection solution containing a labeled antibody on the modified surface of the immunochip, wherein the labeled antibody is an antibody connected with a labeling substance, and the labeled antibody is capable of reacting with the modifying groups and bonding to the modifying groups;
removing the labeled antibody that has not reacted with the modifying groups, and detecting the content of residual labeled antibody on the modified surface; and
identifying the quality of the immunochip according to the content of residual labeled antibody.

2. The method according to claim 1, wherein, the modifying groups are carboxyl groups.

3. The method according to claim 2, wherein, before applying the detection solution containing the labeled antibody on the modified surface of the immunochip, the method further comprises:
performing an activation treatment on the immunochip for increasing the activity of the carboxyl groups modified on the modified surface of the immunochip.

4. The method according to claim 3, wherein, the activation treatment on the immunochip comprises:
placing the immunochip in an activation solution and keeping for a first predetermined time, the activation solution is a mixed solution of a 1-(3-dimethylaminopropyl)-3-ethylcarbodiimide hydrochloride solution and a N-hydroxysuccinimide solution.

5. The method according to claim 4, wherein, the 1-(3-dimethylaminopropyl)-3-ethylcarbodiimide hydrochloride solution and the N-hydroxysuccinimide solution are mixed in a ratio of 1:0.8 to 1:1.2.

6. The method according to claim 4, wherein, the first predetermined time is 10 minutes to 20 minutes.

7. The method according to claim 1, wherein, between applying the detection solution containing the labeled antibody on the modified surface of the immunochip and removing the labeled antibody that has not reacted with the modifying groups, the method further comprises:
placing the immunochip in a predetermined environment and keeping for a second predetermined time.

8. The method according to claim 7, wherein, the second predetermined time is greater than or equal to 8 hours.

9. The method according to claim 7, wherein, the predetermined environment evades the light, and the predetermined environment has an environmental relative humidity of greater than 50%, and an environmental temperature of 2° C. to 6° C.

10. The method according to claim 1, wherein:
the labeling substance is a fluorescent substance, and the labeled antibody is a fluorescent antibody; and
identifying the quality of the immunochip according to the content of residual labeled antibody comprises: identifying the quality of the immunochip according to the fluorescence intensity of the fluorescent substance of the residual fluorescent antibody.

11. The method according to claim 10, wherein: identifying the quality of the immunochip according to the fluorescence intensity of the fluorescent substance of the residual fluorescent antibody fluorescent antibody comprises:
acquiring a fluorescence image of the immunochip;
randomly selecting a plurality of fluorescent regions of a predetermined area from the fluorescent image, and acquiring the fluorescence intensity value of each fluorescent region; and
acquiring an average value of the fluorescence intensity values of the plurality of fluorescent regions, and identifying the quality of the immunochip according to the average value.

12. The method according to claim 1, wherein,
the modified surface includes a quality control area and an experimental area;
applying the detection solution containing the labeled antibody on the modified surface of the immunochip comprises: applying the detection solution containing the labeled antibody on the quality control area.

13. The method according to claim 1, wherein, before applying the detection solution containing the labeled antibody on the modified surface of the immunochip, the method further comprises:
performing an activation treatment on the immunochip for increasing the activity of the carboxyl groups modified on the modified surface of the immunochip.

14. The method according to claim 4, wherein, the 1-(3-dimethylaminopropyl)-3-ethylcarbodiimide hydrochloride solution has a concentration of 6 wt % to 8 wt %; and the N-hydroxysuccinimide solution has a concentration of 1 wt % to 3 wt %.

15. The method according to claim 12, wherein, the quality control area and the experimental area have the same content of modifying groups.

16. The method according to claim 12, wherein, detecting the content of residual labeled antibody on the modified surface comprising fluorescence microscopy, laser confocal microscopy, ultraviolet-visible spectroscopy, gas chromatography, or liquid chromatography.
